(19) United States
(12) Patent Application Publication
(10) Pub. No.: US 2007/0005024 A1
(43) Pub. Date: Jan. 4, 2007

(54) Medical devices having superhydrophobic surfaces, superhydrophilic surfaces, or both

(76) Inventors: Jan Weber, Maple Grove, MN (US); Scott Schewe, Eden Prairie, MN (US); Brian Berg, St. Paul, MN (US)

Correspondence Address: MAYER & WILLIAMS PC, 251 NORTH AVENUE WEST, 2ND FLOOR, WESTFIELD, NJ, 07090, US

(21) Appl. No.: 11/150,082

(22) Filed: Jun. 10, 2005

(51) Int. Cl. A61M 25/00

(52) U.S. Cl. 604/265

(57) ABSTRACT

According to an aspect of the invention, medical devices are provided, which have the following (a) one or more superhydrophobic surface regions, (b) one or more superhydrophilic surface regions having a durometer of at least 40 A, or (c) a combination of one or more superhydrophobic surface regions and one or more superhydrophilic surface regions having a durometer of at least 40 A. Such surfaces are created, for example, to provide reduced resistance to the movement of adjacent materials, including adjacent fluids and solids. Examples of medical device surface regions benefiting from the present invention include, for example, outside and/or inside (luminal) surfaces of the following: vascular catheters, urinary catheters, hydrolyser catheters, guide wires, pullback sheaths, left ventricular assist devices, endoscopes, airway tubes and injection needles, among many other devices.

### TECHNICAL FIELD

The present invention relates to medical devices, and more particularly to medical devices having reduced resistance to movement of fluids and solids.

### BACKGROUND

Medical devices such as catheters, which are adapted for movement through blood vessels or other body lumens, are typically provided with low-friction outer surfaces. If the surfaces of the medical devices are not low-friction surfaces, insertion of the devices into and removal of the devices from the body lumens becomes more difficult, and injury or inflammation of bodily tissue may occur. Low friction surfaces are also beneficial for reducing discomfort and injury that may arise as a result of movement between certain long term devices (e.g., long term catheters) and the surrounding tissue, for example, as a result of patient activity.

One specific example of a catheter that is in common use in medicine today is a balloon catheter for use in balloon angioplasty procedures (e.g., percutaneous transluminal coronary angioplasty or “PCTA”). During these procedures, catheters are inserted for long distances into extremely small vessels and are used to open stenoses of blood vessels by balloon inflation. Low friction surfaces are desired to reduce the likelihood of tissue injury and device obstruction in such applications.

In addition, these applications require catheters that have extremely small diameters, because catheter diameter limits the treatable vessel size. Smaller catheter diameters, however, lead to smaller fluid conduits, for example, the fluid conduits which are used to transport inflation fluid to and from the balloons. Unfortunately, as one makes such conduits smaller, the flow resistance that is encountered increases dramatically. For example, for laminar flow in a hollow cylinder, the flow resistance is inversely proportional to the fourth power of the diameter. Furthermore, cells, cell fragments, proteins, DNA or other high molecular weight biomolecules that are transported through small conduits may experience damage due to the high shear forces that are encountered with small fluid conduits. Still another problem arising from flow in small conduits is that, due to the parabolic shaped flow-distribution that is encountered (see the upper no-slip surface in FIG. 1, described below), an initial small and defined liquid volume may spread out over the length of the conduit which makes precise dosing less accurate when using long conduits.

### SUMMARY OF THE INVENTION

According to an aspect of the invention, medical devices are provided, which have the following: (a) one or more superhydrophobic surface regions, (b) one or more superhydrophilic surface regions having a durometer of at least 40 A, or (c) a combination of one or more superhydrophobic surface regions and one or more superhydrophilic surface regions having a durometer of at least 40 A. Such surfaces are created, for example, to provide reduced resistance to the movement of adjacent materials, including adjacent fluids and solids.

Examples of medical device surface regions benefiting from the present invention include, for example, outside and/or luminal surfaces of the following devices: vascular catheters, urinary catheters, hydrolyser catheters, guide wires, pullback sheaths, left ventricular assist devices, endoscopes, airway tubes and injection needles, among many other devices.

An advantage of the present invention is that medical devices may be provided which display reduced friction when they are moved along the surface of another body, for example, the walls of a blood vessel or another bodily lumen or a surface of a medical article.

Another advantage of the present invention is that medical devices may be provided which encounter less resistance to fluid flow along their surfaces.

These and other aspects, embodiments and advantages of the present invention will become immediately apparent to those of ordinary skill in the art upon review of the Detailed Description and Claims to follow.

### BRIEF DESCRIPTION OF O THE DRAWING

FIG. 1 is a schematic diagram illustrating the concepts of slip and no slip at the fluid boundary.

FIGS. 2A and 2B are schematic diagrams illustrating relevant parameters in evaluating surface roughness.

FIG. 3A is a schematic, longitudinal, cross-sectional view of the distal end of a balloon catheter as it is advanced over a guidewire, in accordance with an embodiment of the present invention. FIGS. 3B and 3C are schematic, axial, cross-sectional views of the balloon catheter of FIG. 3A, taken along planes B-B and C-C, respectively.

FIG. 4A is a schematic, longitudinal, cross-sectional view of the distal end of a sheath-based catheter, as it is advanced over a guidewire, in accordance with an embodiment of the present invention. FIG. 4B is a schematic, axial, cross-sectional view of the balloon catheter of FIG. 4A, taken along plane B-B.

### DETAILED DESCRIPTION OF THE INVENTION

The present invention provides medical devices which have reduced resistance to movement of adjacent materials, including both fluids and solids.

In this regard, resistance to movement between a medical device and an adjacent solid may be reduced in either wet or dry conditions by providing the medical device (as well as the adjacent solid, if feasible) with a low energy surface. Such surfaces are typically hydrophobic surfaces, which may be defined as a surface having a static water contact angle that is greater than 90°.

According to an aspect of the present invention, medical devices are provided which have one or more superhydrophobic surface regions (also sometimes referred to as superhydrophobic surfaces, ultrahydrophobic surface regions, or ultrahydrophobic surfaces). For purposes of the present invention, a superhydrophobic surface is one that displays dynamic (receding or advancing) water contact angles above 145° (e.g., ranging from 145° to 150° to 155° to 160° to 165° to 170° to 175° to 180°). In particularly beneficial embodiments, both the receding and the advancing water contact angles are above 145°.

The Wilhelmy plate technique is a suitable technique for measuring the dynamic contact angles for various surfaces, including the superhydrophobic surfaces that are formed in conjunction with the present invention. This technique is performed with a solid sample, typically a rectangular plate or some other regular shape such as a cube, round rod, square rod, tube, etc. To the extent that the medical device of interest is not of sufficiently regular geometry to allow its surface to be tested directly using this technique, a sample of regular geometry, which is provided with a surface using the same process that is used to provide the medical device surface, may be tested so as to infer the dynamic contact angles of the device.

The Wilhelmy plate technique is performed using a tensiometer. The solid sample is immersed into and withdrawn out of a liquid (i.e., water) while simultaneously measuring the force acting on the solid sample. Advancing and receding contact angles can then be determined from the obtained force curve using well known calculations. The advancing contact angle is the contact angle that is measured as the sample is immersed in the liquid, whereas the receding contact angle is the contact angle that is measured as the sample is removed from the liquid. A typical way of enhancing hydrophobicity is to employ materials with low surface energy, such as fluorocarbon polymers. However, even fluorocarbon materials yield water contact angles that are only around 120° or so. Nevertheless, surfaces with substantially greater water contact angles do exist in nature, and they have been created in the laboratory. In general, in addition to being formed from low surface energy (inherently hydrophobic) materials, these surfaces have been shown to have microscale and/or nanoscale surface texturing. A superhydrophobic biological material commonly referred to in the literature is the lotus leaf, which has been observed to be textured with 3-10 micron hills and valleys, upon which are found nanometer sized regions of hydrophobic material.

Consequently, medical device surfaces in accordance with certain aspects of the present invention have the following surface characteristics: (a) a peak roughness average, or Rₚₘ, between 100 nm and 5 micrometers, (b) a mean spacing between peaks, or Sₘ, that is >10 times the Rₚₘ value, and (c) a surface material having a low surface energy (i.e., the material is inherently hydrophobic, meaning that the material displays a contact angle that ranges from 90° to 100° to 110° to 115° to 120°, independent of surface roughness). These surface characteristics may exist independent of or in addition to a dynamic water contact angle above 145°.

Sₘ is defined as the mean spacing between peaks, with a peak defined relative to the mean line of the surface. For any given peak width, a peak must cross above the mean line and then back below it (see, e.g., peak width S₁ in FIG. 2A). If the width of each peak is denoted as Sᵢ, then the mean spacing is the average width of a peak among N peaks measured is as follows:

$${S}_{m} = ( 1 / N ) {\sum }_{n = 1}^{N} {S}_{n} .$$

Peak roughness, or Rₚ, is the height of the highest peak in the roughness profile that is detected over the evaluation length. See, e.g., Rₚ in FIG. 2B, which is the height of the highest peak measured over the evaluation length p₁. Peak roughness average, or Rₚₘ, is the average peak roughness measured over M evaluation segments. Rₚₘ is expressed mathematically as

$${R}_{pm} = \frac{1}{M} {\sum }_{i = 1}^{M} {R}_{pi} .$$

Equipment is commercially available for the routine measurement of Sₘ and Rₚₘ, for example, the Portable Surface Roughness Model TR200 from Micro Photonics Inc., 4972 Medical Center Circle, Allentown, Pa., USA. A much more detailed surface topography can be obtained using the Micromeasure 3D Non-Contact Profilometry System, also available from Micro Photonics.

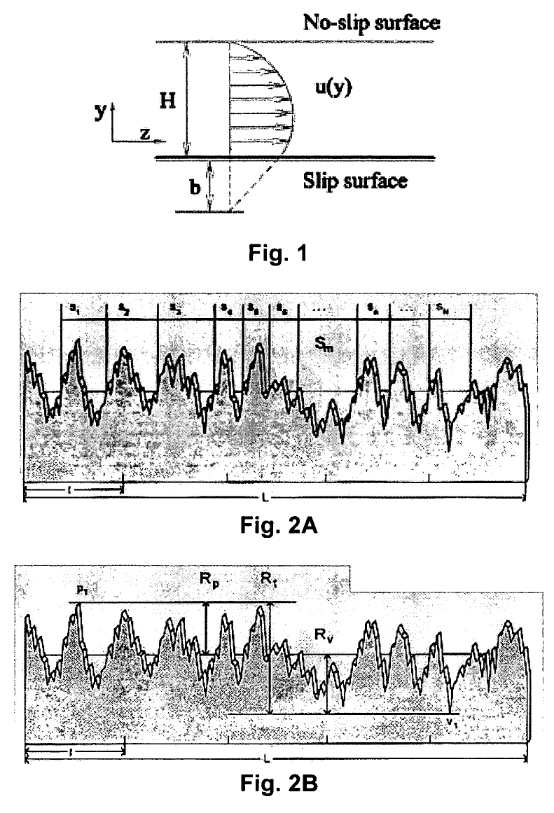

Fluid flow adjacent to superhydrophobic surfaces has been observed to display interesting characteristics, including wall slip. Without wishing to be bound by theory, the concept of wall slip vs. no wall slip may be understood with reference to FIG. 1, which illustrates an upper no-slip surface and a lower slip surface. The velocity of a fluid flowing in the z-direction within space H between these surfaces depends upon the distance in the y direction that exists between the fluid and the surfaces. This velocity u is represented by the rightward-pointing arrows. As can be seen, the velocity u of the fluid at the no-slip surface is zero, whereas the velocity u at the slip surface is not. Mathematically, slip velocity u at the surface is proportional to the shear rate experienced by the fluid at the wall (du/dy) multiplied by the slip length b. (The slip length b may be defined as the distance behind the slip boundary at which the flow velocity extrapolates to zero.)

The no-slip condition is typically accepted as the proper boundary condition at a solid-liquid interface. While fluids are generally believed to have some degree of slip at the wall, the slip lengths are generally only on the order of molecular sizes such that they are significant only in channels of extremely small length scale. With superhydrophobic surfaces, on the other hand, slip lengths on the order of tens and even hundreds of microns have been reported for aqueous solutions. See, e.g., Jia Ou et al, “Laminar drag reduction in microchannels using ultrahydrophobic surfaces, Physics Of Fluids, Vol. 16, No. 12, December 2004; Chang-Jin “C J” Kim and Chang-Hwan Choi, “Nano-engineered Low-friction Surface for Liquid Flow,” Program of the 6th KSME-JSME Thermal and Fluids Engineering Conference, Mar. 20-23, 2005, Jeju, Korea; E. Lauga and H. Stone, “Effective slip in pressure-driven Stokes flow,” J. Fluid Mech. (2003), vol. 489, pp. 55-77.

Slip lengths for surfaces, including the superhydrophobic surfaces that are formed in conjunction with the present invention, may be measured using micron-resolution particle image velocimetry as described in D C Threteway and C D Meinhart, “Apparent fluid slip at hydrophobic microchannel walls” Physics Of Fluids, Volume 14, Number 3, March 2002, pp. L9-L12. A more conventional method is to measure flow rate through a fluid channel and directly calculate the slip length from the increase of flow rate that is observed, as compared to that expected under conditions of laminar flow with zero slip-length at the wall. For example, see the above Lauga and Stone reference, in which an experimental flow cell is described that measures the pressure drop resulting from the laminar flow of water through a rectangular microchannel. The lower wall of the microchannel is designed to be interchangeable, making it possible to perform drag reduction measurements on various surfaces. Techniques of this type may also be desirable for the measurement of wall slip in other regular geometries, for example, small tubes and small annular channels, such as those found within catheters (note that no optical access to the space is required using such techniques). To the extent that a medical device in accordance with the present invention is not sufficiently regular to conduct wall slip measurements on the device itself, a sample of regular geometry, which is provided with a surface using the same process that is used to provide the medical device surface, may be tested so as to infer the slip length associated with the device surface. In this regard, see also the above Ou et al. reference, in which the effective slip length of a surface is measured via torque measurement using a commercial cone-and-plate rheometer system. Slip lengths in accordance with the invention may vary widely with exemplary ranges being 10 to 25 to 50 to 100 microns or more.

One consequence of slip at the wall is that resistance to fluid flow is reduced. As the width of the fluid conduit of interest (e.g., the diameter for a tubular conduit, the distance between the inner and outer cylindrical elements of an annular conduit, etc.) approaches the slip length, the effects of wall slip can become substantial. For example, the annular inflation lumens for some balloon catheters have a wall-to-wall spacing of approximately 0.180 mm, possibly going to 0.160 mm or even lower in the near future. These distances are on the same order as the superhydrophobic slip lengths described above.

In addition to increasing flow for a given pressure drop, wall slip also has the effect of reducing shear between the wall and the boundary fluid layer, which may result in less damage to high-molecular-weight and particulate biologicals (e.g., proteins, DNA, cells, cell fragments, etc.) and may reduce the tendency of an initial small and defined liquid volume to spread out as it travels down the length of the conduit.

Another way of reducing resistance to movement between a medical device and an adjacent solid under wet conditions is to provide the medical device with a high energy surface. Such surfaces may be characterized, for example, as hydrophilic, which may be defined as a surface having a water contact angle that is less than or equal to 90°.

According to another aspect of the present invention, medical devices are provided which have one or more superhydrophilic surfaces. A surface with a static water contact angle of 20° or less (e.g., ranging from 20° to 10° to 5° to 2° to 1° to 0.50 to 0°) is considered to be a superhydrophilic surface for purposes of the present invention. Moreover, unlike hydrogel surfaces, superhydrophilic surfaces for use in the medical devices of the invention are hard, even when immersed in water, for example, having a Durometer/Shore Hardness of at least 40 A.

Medical devices benefiting from superhydrophobic surfaces, superhydrophilic surfaces, or both, include a variety of implantable and insertable medical devices (referred to herein as “internal medical devices”). Examples of such medical devices include, devices involving the delivery or removal of fluids (e.g., drug containing fluids, pressurized fluids such as inflation fluids, bodily fluids, contrast media, hot or cold media, etc.) as well as devices for insertion into and/or through a wide range of body lumens, including lumens of the cardiovascular system such as the heart, arteries (e.g., coronary, femoral, aorta, iliac, carotid and vertebro-basilar arteries) and veins, lumens of the genitourinary system such as the urethra (including prostatic urethra), bladder, ureters, vagina, uterus, spermatic and fallopian tubes, the nasolacrimal duct, the eustachian tube, lumens of the respiratory tract such as the trachea, bronchi, nasal passages and sinuses, lumens of the gastrointestinal tract such as the esophagus, gut, duodenum, small intestine, large intestine, rectum, biliary and pancreatic duct systems, lumens of the lymphatic system, the major body cavities (peritoneal, pleural, pericardial) and so forth.

Non-limiting, specific examples of internal medical devices include vascular devices such as vascular catheters (e.g., balloon catheters), including balloons and inflation tubing for the same, hydrolyser catheters, guide wires, pullback sheaths, filters (e.g., vena cava filters), left ventricular assist devices, total artificial hearts, injection needles, drug delivery tubing, drainage tubing, gastroenteric and colonoscopic tubing, endoscopic devices, endotracheal devices such as airway tubes, devices for the urinary tract such as urinary catheters and ureteral stents, and devices for the neural region such as catheters and wires. Many devices in accordance with the invention have one or more portions that are cylindrical in shape, including both solid and hollow cylindrical shapes.

Devices in accordance with the present invention may have a single superhydrophobic surface region or multiple superhydrophobic surface regions.

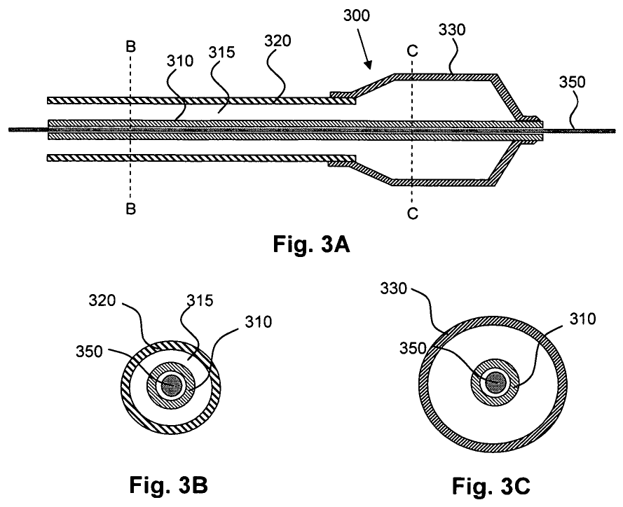

Various specific embodiments of the present invention will now be described in conjunction with FIGS. 3A-3C, in which the distal end of a guidewire-balloon catheter system is illustrated. As seen from the schematic, longitudinal cross-section of FIG. 3A, this system includes a guidewire 350, which passes through a lumen formed by an inner tubular member 310. Also shown is an outer tubular member 320, which, along with inner tubular member 310, forms an annular inflation lumen 315 that provides for the flow of inflation fluid into balloon 330. FIGS. 3B and 3C are schematic, axial, cross-sectional views of the balloon catheter of FIG. 3A, taken along planes B-B and C-C, respectively.

In such a system, it may be desirable to decrease the friction at various locations including (a) between the guidewire 350 and the vasculature through which it is advanced, (b) between the inside surface of the member that forms the guidewire lumen of the catheter (e.g., inner tubular member 310) and the outside surface of the guidewire 350 over which it is passed, (c) between the inside surface of the balloon 330 and the outside surface of the inner tubular member 310, (d) between the outside surface of the balloon 330 and the vasculature, and/or (e) between the outside surface of the outer tubular member 320 and the vasculature. For this purpose, such surfaces may be rendered superhydrophobic or superhydrophilic in accordance with the present invention, for example, using techniques such as those described herein.

Note that it may be desirable treat only a portion of a given surface. As a specific example, balloons may be advanced into the vasculature while in a folded configuration, in which case the exposed balloon surface may be rendered superhydrophobic or superhydrophilic in conjunction with the present invention. It may be desirable, however, to the avoid so-treating the non-exposed (folded) balloon surface, thereby allowing the balloon to better engage surrounding tissue (or a surrounding stent) upon deployment of the balloon and decreasing the likelihood of slippage. Where the balloon is configured to refold upon deflation along the same lines that it was folded prior to inflation, a substantially superhydrophobic or superhydrophilic surface will again be presented to the vasculature, assisting with balloon withdrawal.

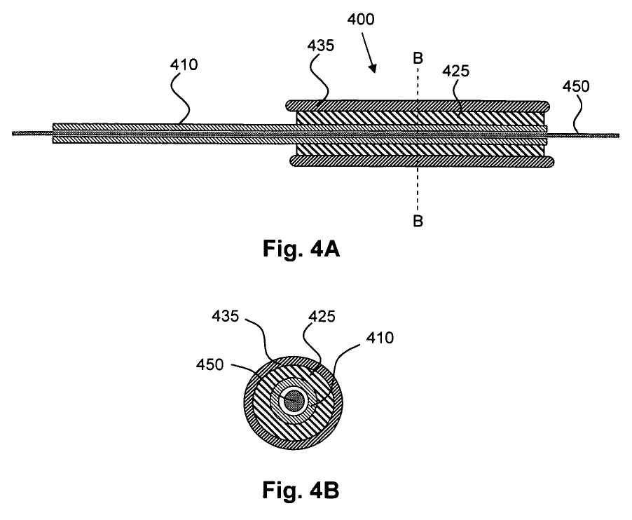

The distal end of another guidewire-catheter system will now be described with reference to FIGS. 4A and 4B. As seen from these figures, this system includes a guidewire 450, which passes through a lumen formed by a tubular member 410. Disposed around the distal end of the tubular member 410 is a self-expanding stent 425, which may be formed from any of a number of biodegradable and biostable materials known in the art, including various polymeric and metallic materials, suitable members of which may be selected from polymeric and metallic materials listed further below. Self-expanding stent 425 is in a radially contracted state as shown, exerting a radially outward force against sheath 435, which maintains the stent 425 in the contracted state. Upon being advanced to a desired site within a subject, the stent 425 is deployed by pulling back the sheath 435 in a distal direction.

As with the system illustrated in FIGS. 3A-3C, it may be desirable to decrease the friction at various locations in this system. For example, it may be desirable to decrease the friction (a) between the guidewire 450 and the vasculature through which it is advanced, (b) between the inside surface of the member that forms the guidewire lumen of the catheter (e.g., tubular member 410) and the outside surface of the guidewire 450 over which it is passed, (c) between the outside surface of the stent 425 and the sheath 435 which is withdrawn distally over the outside surface of the stent 425, and/or (d) between the outside surface of the sheath 435 and the vasculature though which it is advanced. For this purpose, one or more of these surfaces may be rendered superhydrophobic or superhydrophilic in accordance with the present invention, for example, using techniques such as those described herein.

As previously noted, it is also desirable to decrease the resistance to fluid flow that is encountered, for example, by inflation fluid as it proceeds down the length of the catheter to the balloon (upon inflation) and back (upon deflation). For this purpose, the fluid-contacting surface(s) of the conduit through which the inflation fluid travels may be rendered superhydrophobic. (The inflation fluid may be an aqueous or non-aqueous liquid, and the degree of wall slip encountered by the fluid may be among the criteria for inflation fluid selection, if desired.) Moreover, by providing the outer surface of the catheter with a superhydrophobic outer surface, resistance to blood flow between the outer surface of the catheter and the inside of the vessel may be substantially reduced in very narrow passages, for example, those encountered in conjunction with chronic total occlusions.

As indicated above, in addition to being formed from a low surface energy material (e.g., an inherently hydrophobic material), superhydrophobic surfaces generally have an associated surface roughness. Examples of low surface energy materials include fluorocarbon materials (i.e., materials containing molecules having C—F bonds), for instance, fluorocarbon homopolymers and copolymers such as polytetrafluoroethylene (PTFE), fluorinated ethylene propylene (FEP), ethylene tetrafluoroethylene (ETFE), ethylene chloro-trifluoroethylene (ECTFE), perfluoro-alkoxyalkane (PFA), poly(chloro-trifluoro-ethylene) (CTFE), perfluoro-alkoxyalkane (PFA), and poly(vinylidene fluoride) (PVDF), among many others.

Many techniques are available for creating superhydrophobic surfaces, a few of which are described herein.

In some embodiments, a substrate material having a low surface energy (i.e., an inherently hydrophobic material) may be textured to produce a superhydrophobic surface. For instance, a low surface energy substrate material (e.g., a fluorocarbon layer) may be textured using techniques such as those described below.

Alternatively, a substrate material may be textured (e.g., using techniques such as those described below), followed by application of a coating of a low surface energy (i.e., inherently hydrophobic) material that is sufficiently thin to reflect at least some of the contours of the textured surface.

In this way, a wide range of substrate materials may be employed for the practice invention, suitable examples of which may be selected, for example, from the various substrate materials set forth below.

One example of a technique for depositing thin layers of low surface energy (i.e., inherently hydrophobic) materials is hot-filament CVD (HFCVD), also known as pyrolytic or hot-wire CVD. HFCVD allows objects of complex shape and nanoscale feature size to be conformally coated. For example, the conformal nature of HFCVD has been demonstrated to allow carbon nanotubes to be “shrink-wrapped”. Using hot filaments to drive the gas phase chemistry allows linear polymers to be deposited, as opposed to highly crosslinked networks such as those encountered with other techniques such as plasma enhanced CVD. This technique can be used to deposit ultrathin layers of a variety of polymers, including low surface energy polymers such as polytetrafluoroethylene. Besides being able to deposit ultrathin layers, this technique is advantageous in that the object to be coated remains at room temperature. For further information, see, e.g., United States Patent Application No. 2003/0138645 to Gleason et al.; K. K. S. Lau et al., “Hot-wire chemical vapor deposition (HWCVD) of fluorocarbon and organosilicon thin films,” Thin Solid Films, 395 (2001) pp. 288-291; Lau K K S and Gleason K K. “Pulsed plasma enhanced and hot filament chemical vapor deposition of fluorocarbon films” J. Fluorine Chem., 2000, 104, 119-126; and Lau K K S et al., “Structure and morphology of fluorocarbon films grown by hot filament chemical vapor deposition”. Chem. of Mater., 2000, 12, 3032-3037.

Examples of techniques by which surfaces may be textured include, for example, laser ablation techniques such as laser induced plasma spectroscopy (LIPS) structuring. A laser technique for providing surface texturing is described, for example, in Wong, W. et al, “Surface structuring of poly(ethylene terephthalate) by UV excimer laser,” Journal of Materials Processing Technology 132 (2003) 114-118. Techniques for forming textured surfaces on one or more components of a medical device by laser treatment at high fluence and/or by plasma treatment are described in U.S. Ser. No. 11/045,955 filed Jan. 26, 2005 and entitled “Medical Devices and Methods of Making the Same.”

Other methods for surface roughening are based on lithographic techniques in which a patterned mask is formed over the material to be textured, and the material is subsequently etched through apertures in the mask. Lithographic techniques include optical lithography, ultraviolet and deep ultraviolet lithography, and X-ray lithography. One process, known as columnated plasma lithography, is capable of producing X-rays for lithography having wavelengths on the order of 10 nm. For an example of the use of photolithographic techniques to form surface texturing, see, e.g., Jia Ou et al, “Laminar drag reduction in microchannels using ultrahydrophobic surfaces,” Physics Of Fluids, Vol. 16, No. 12, December 2004. In this article, pressure drop reductions up to 40% and apparent slip lengths larger than 20 microns are obtained for the laminar flow of water through microchannels having ultrahydrophobic surfaces.

Still other methods for forming textured surfaces, including nanotextured surfaces, are described in U.S. Ser. No. 11/007,867 entitled “Medical Devices having Nanostructured Regions for Controlled Tissue Biocompatibility and Drug Delivery.” These methods include methods in which textured regions are formed by: (a) providing a precursor region comprising a first material that is present in distinct phase domains within the precursor region; and (b) subjecting the precursor region to conditions under which the first material is either reduced in volume or eliminated from the precursor region (e.g., because the first material is preferentially sublimable, evaporable, combustible, dissolvable, etc.), thereby forming a textured region. Examples include alloys that contain dissolvable/etchable metallic phase domains (e.g. Zn, Fe, Cu, Ag, etc.) along with one or more substantially non-oxidizing noble metals (e.g., gold, platinum, titanium, etc.). Further details concerning dealloying can be found, for example, in J. Erlebacher et al., “Evolution of nanoporosity in dealloying,” Nature, Vo. 410, 22 March 2001, 450-453; A. J. Forty, “Corrosion micromorphology of noble metal alloys and depletion gilding,” Nature, Vol. 282, 6 Dec. 1979, 597-598; and R. C. Newman et al., “Alloy Corrosion,” MRS Bulletin, July 1999, 24-28.

In other embodiments, a coating is created over an underlying substrate material, which provides both the surface roughness and the low surface energy characteristics that are generally associated with superhydrophobic surfaces. Such coatings may be of single or multiple layer construction and may be applied over a wide variety of substrate materials. Various specific techniques for forming such coatings will now be described.

One specific example of a situation where a superhydrophobic coating is provided over an underlying substrate is described in P. Favia et al., “Deposition of super-hydrophobic fluorocarbon coatings in modulated RF glow discharges,” Surface and Coatings Technology, 169-170 (2003) 609-612. Favia et al. have reported the deposition of superhydrophobic coatings in modulated RF glow discharges fed with tetrafluoroethylene. These coatings are characterized as having a high degree of fluorination and as having ribbon-like randomly distributed surface microstructures, which have feature sizes on the order of a micron. The combined high fluorination degree and surface texture roughness was reported to lead to superhydrophobic behavior, as attested by water contact angle values of 150-165°.

Textured surfaces may also be created using sol-gel techniques. In a typical sol-gel process, precursor materials are subjected to hydrolysis and condensation (also referred to as polymerization) reactions to form a colloidal suspension, or “sol”. Examples of precursors include inorganic metallic and semi-metallic salts, metallic and semi-metallic complexes/chelates (e.g., metal acetylacetonate complexes), metallic and semi-metallic hydroxides, organometallic and organo-semi-metallic alkoxides (e.g., metal alkoxides and silicon alkoxides), among others. As can be seen from the simplified scheme below, the sol-forming reaction is basically a ceramic network forming process (from G. Kickelbick, “Concepts for the incorporation of inorganic building blocks into organic polymers on a nanoscale” Prog. Polym. Sci., 28 (2003) 83-114):

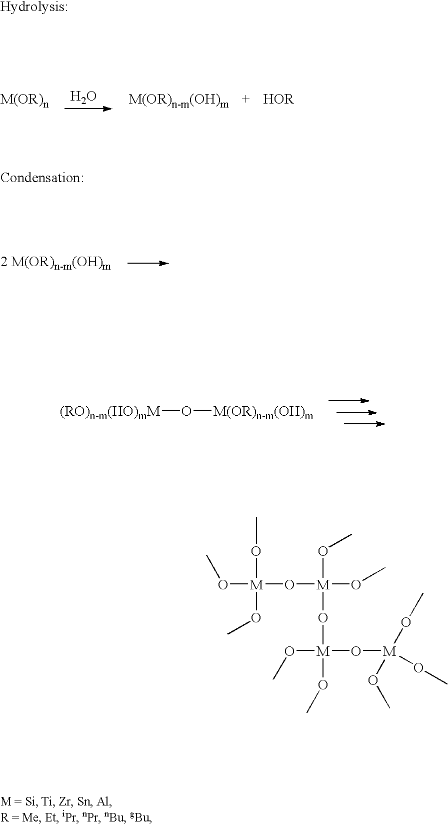

A textured layer may be produced by applying a sol onto a substrate, for example, by spray coating, coating with an applicator (e.g., by roller or brush), spin-coating, dip-coating, and so forth. As a result, a “wet gel” is formed. The wet gel is then dried. If the solvent in the wet gel is removed under supercritical conditions, a material commonly called an “aerogel” is obtained. If the gel is dried via freeze drying (lyophilization), the resulting material is commonly referred to as a “cryogel.” Drying at ambient temperature and ambient pressure leads to what is commonly referred to as a “xerogel.” Other drying possibilities are available including elevated temperature drying (e.g., in an oven), vacuum drying (e.g., at ambient or elevated temperatures), and so forth. The porosity, and thus surface texture, of the gel can be regulated in a number of ways, including, for example, varying the solvent/water content, varying the aging time (e.g., the time before addition of an aqueous solution to a metal organic solution), varying the drying method and rate, and so forth. Further information concerning sol-gel materials can be found, for example, in Viitala R. et al., “Surface properties of in vitro bioactive and non-bioactive sol-gel derived materials,” Biomaterials, 2002 Aug; 23(15):3073-86.

The production of hydrophobic sol-gels with high contact angles have been reported through the use of various organosilane compounds. See, e.g., A. V. Rao et al., “Comparative studies on the surface chemical modification of silica aerogels based on various organosilane compounds of the type RₙSiX₄₋ₙ,” Journal of Non-Crystalline Solids 350 (2004) 216-223, which reports the surface chemical modification of silica aerogels using various precursors and co-precursors based on mono-, di-, tri- and tetrafunctional organosilane compounds. The chemically modified hydrophobic silica aerogels are produced by (i) co-precursor, and (ii) derivatization methods. The co-precursor method resulted in aerogels with higher contact angle (approx. 136°) whereas a lower contact angle (approx. 120°) arose using the derivatization method. Using the coprecursor, aerogels with contact angles as high as 175° were obtained.

In other embodiments of the invention, once a gel layer of suitable porosity is formed, it is provided with a thin low surface energy (i.e., inherently hydrophobic) layer, for example, a fluorocarbon layer, such as those described elsewhere herein.

Another example where a multilayer coating process is employed to provide a superhydrophobic surface is described in K. K. S. Lau et al., “Superhydrophobic Carbon Nanotube Forests” Nanoletters 3, 1701 (2003). In this work, stable, superhydrophobic surfaces are created using the nano-scale roughness inherent in a vertically aligned carbon nanotube “forest.” The nanotube layer is deposited using a plasma enhanced chemical vapor deposition (PECVD) technique that consists of forming discrete nickel catalyst islands on a substrate and subsequently growing nanotubes from these catalyst islands in a DC plasma discharge. A thin, conformal polytetrafluoroethylene layer is then applied onto the carbon nanotubes using the HFCVD process. More particularly, using an array of stainless steel filaments resistively heated to 500° C., hexafluoropropylene oxide (HFPO) gas is thermally decomposed to form difluorocarbene (CF₂) radicals, which polymerize into PTFE on the nanotube layer, which is kept at room temperature. An initiator, e.g., perfluorobutane-1-sulfonyl fluoride, is used to promote the polymerization process. The advancing and receding contact angles of the resulting surface are 170° and 160°, respectively.

Other multilayer techniques for forming ultrahydrophobic surface coatings include the use of layer-by-layer techniques, in which a wide variety of substrates may be coated using charged materials via electrostatic self-assembly. In the layer-by-layer technique, a first layer having a first surface charge is typically deposited on an underlying substrate, such as one of those described above, followed by a second layer having a second surface charge that is opposite in sign to the surface charge of the first layer, and so forth. The charge on the outer layer is reversed upon deposition of each sequential layer.

Layer-by-layer techniques generally employ charged polymer species, including those commonly referred to as polyelectrolytes. Specific examples of polyelectrolyte cations (also known as polycations) include protamine sulfate polycations, poly(allylamine) polycations (e.g., poly(allylamine hydrochloride) (PAH)), polydiallyldimethylammonium polycations, polyethyleneimine polycations, chitosan polycations, gelatin polycations, spermidine polycations and albumin polycations, among many others.

Specific examples of polyelectrolyte anions (also known as polyanions) include poly(styrenesulfonate) polyanions (e.g., poly(sodium styrene sulfonate) (PSS)), polyacrylic acid polyanions, sodium alginate polyanions, eudragit polyanions, gelatin polyanions, hyaluronic acid polyanions, carrageenan polyanions, chondroitin sulfate polyanions, and carboxymethylcellulose polyanions, among many others.

Surface roughness may be created in these techniques by depositing one or more layers of particles. A variety of particles are available for this purpose including, for example, carbon, ceramic and metallic particles, which may be in the form of plates, cylinders, tubes, and spheres, among other shapes. Specific examples of plate-like particles include synthetic or natural phyllosilicates including clays and micas (which may optionally be intercalated and/or exfoliated) such as montmorillonite, hectorite, hydrotalcite, vermiculite and laponite. Specific examples of tubes and fibers include single-wall, so-called “few-wall,” and multi-wall carbon nanotubes, vapor grown carbon fibers, alumina fibers, titanium oxide fibers, tungsten oxide fibers, tantalum oxide fibers, zirconium oxide fibers, silicate fibers such as aluminum silicate fibers, and attapulgite clay. Specific examples of further particles include fullerenes (e.g., “Buckey balls”), silicon oxide (silica) particles, aluminum oxide particles, titanium oxide particles, tungsten oxide particles, tantalum oxide particles, and zirconium oxide particles.

In some embodiments, charged particle layers are introduced as part of the layer-by-layer process. Certain particles, such as clays, have an inherent surface charge. On the other hand, surface charge may be provided, if desired, by attaching species that have a net positive or negative charge to the particles, for example by adsorption, covalent bonding, and so forth.

One specific layer-by-layer technique for forming superhydrophobic surfaces on underlying substrates is described in L. Zhai et al., “Stable Superhydrophobic Coatings from Polyelectrolyte Multilayers,” Nano Letters, 2004, Vol. 4, No. 7, 1349-53. In this study the lotus leaf structure is mimicked by creating a porous, honeycomb-like polyelectrolyte multilayer surface, overcoated with silica nanoparticles. This structure is then further coated with a semifluorinated silane. More specifically, this reference describes a process in which multilayers are assembled from poly(allylamine hydrochloride) (PAH) and poly(acrylic acid) (PAA) with the PAH dipping solution at a pH of 8.5 and the PAA dipping solution at a pH of 3.5. A resulting 100.5-bilayer-thick PAH/PAA coating is then subject to a staged low pH treatment protocol to form pores on the order of 10 microns and having a honeycomb-like structure on the surface. To mimic the lotus leaf effect, this micron scale surface is further provided with nanoscale surface texture. Nanoscale texture is introduced by depositing 50 nm SiO₂ nanoparticles onto the surface by alternating dipping of the substrate into an aqueous suspension of negatively charged nanoparticles, followed by dipping in aqueous PAH solution, followed by a final dipping of the substrate into the nanoparticle suspension. The surface is then modified by a chemical vapor deposition of (tridecafluoro-1,1,2,2-tetrahydrooctyl)-1-trichlorosilane (semifluorinated silane) followed by heating at 180° C. to remove unreacted semifluorinated silane. The resulting surface demonstrated advancing and receding water contact angles which were in excess of 160°.

Another specific layer-by-layer technique for forming superhydrophobic surfaces on underlying substrates is described in R. M. Jisr et al., “Hydrophobic and Ultrahydrophobic Multilayer Thin Films from Perfluorinated Polyelectrolytes,” Angew. Chem. Int. Ed. 2005, 44, 782-785. The polyelectrolytes employed are poly(diallyidimethylammonium) (PDADMA), 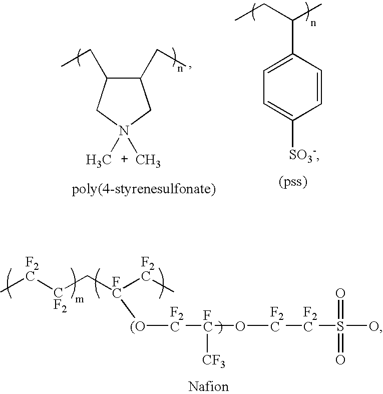
and a polycation synthesized from poly(vinylpyridine) and a fluorinated alkyl iodide, 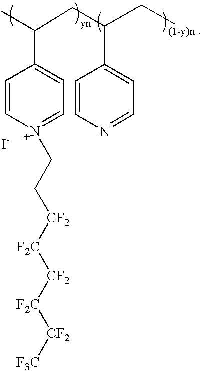
Attapulgite, a negatively charged clay mineral with a needlelike morphology, is used to form the particulate layers. As is typical of layer-by-layer processes, polyelectrolytes are deposited under ambient conditions using dilute solutions/dispersions, in this case in methanol. Three groups of bilayer pairs are deposited. The first, adjacent to the substrate, consists of several PDADMA/PSS bilayers. This is followed by additional bilayers of clay particles and PDADMA, which produces surface roughness, and is in turn followed by bilayers of fluorinated polyelectrolytes, specifically the nafion and PFPVP. No annealing steps are required. The resulting surface had advancing and receding water contact angles in excess of 140°, even after 2 months of immersion in water.

Jisr et al. further demonstrate that non-hydrogel, superhydrophilic surfaces can readily be created by application of a hydrophilic polyelectrolyte, even when deposited over a superhydrophobic structure. Specifically, the above ultrahydrophobic coating was transformed into an ultrahydrophilic surface by coating it with 2.5 additional layer pairs of PAA-co-PAEDAPS and PFPVP. The PAA-co-PAEDAPS is a statistical copolymer of 75 mol % poly(acrylic acid) and 25 mol % poly((3-[2-(acrylamido)ethyldimethylammonio]-propane sulfonate), a hydrophilic zwitterion. The resulting surface had a contact angle of 0° (too small to measure).

Unlike other known superhydrophilic materials such as hydrogels, superhydrophilic materials made using layer-by-layer techniques can be hard, for example, having a durometer value similar to elastomeric polymers used to produce catheter tubes (e.g., 40 A or more, in some instances).

It is noted that certain of the above techniques are particularly well adapted to forming superhydrophobic and superhydrophilic surfaces over the interior surfaces of medical devices and medical device components (e.g., tubes, etc.), including sol-gel layer-by-layer techniques, layer-by-layer techniques and HFCVD.

As previously indicated, substrate materials for use in the invention vary widely and may be selected from (a) organic materials (e.g., materials containing 50 wt % or more organic species) such as polymeric materials and (b) inorganic materials (e.g., materials containing 50 wt % or more inorganic species), such as metallic materials (e.g., metals and metal alloys) and non-metallic materials (e.g., including carbon, semiconductors, glasses and ceramics, which may contain various metal- and non-metal-oxides, various metal- and non-metal-nitrides, various metal- and non-metal-carbides, various metal- and non-metal-borides, various metal- and non-metal-phosphates, and various metal- and non-metal-sulfides, among others).

Specific examples of non-metallic inorganic materials may be selected, for example, from materials containing one or more of the following: metal oxides, including aluminum oxides and transition metal oxides (e.g., oxides of titanium, zirconium, hafnium, tantalum, molybdenum, tungsten, rhenium, and iridium); silicon; silicon-based ceramics, such as those containing silicon nitrides, silicon carbides and silicon oxides (sometimes referred to as glass ceramics); calcium phosphate ceramics (e.g., hydroxyapatite); carbon; and carbon-based, ceramic-like materials such as carbon nitrides.

Specific examples of metallic inorganic materials may be selected, for example, from metals (e.g., biostable metals such as gold, platinum, palladium, iridium, osmium, rhodium, titanium, tantalum, tungsten, and ruthenium, and bioresorbable metals such as magnesium and iron), metal alloys comprising iron and chromium (e.g., stainless steels, including platinum-enriched radiopaque stainless steel), alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N) and alloys comprising cobalt, chromium, tungsten and nickel (e.g., L605), alloys comprising nickel and chromium (e.g., inconel alloys), and bioabsorbable metal alloys, such as alloys of magnesium or iron in combination with Ce, Ca, Zn, Zr and/or Li.

Substrate materials containing polymers and other high molecular weight materials may be selected, for example, from substrate materials containing one or more of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polyether-block co-polyamide polymers (e.g., Pebax® resins), polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, vinyl aromatic polymers and copolymers such as polystyrenes, styrene-maleic anhydride copolymers, vinyl aromatic-hydrocarbon copolymers including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); polyesters including polyethylene terephthalates, polybutylene terephthalates and aliphatic polyesters such as polymers and copolymers of lactide (which includes lactic acid as well as d-, l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and further copolymers of the above.

Although various embodiments of the invention are specifically illustrated and described herein, it will be appreciated that modifications and variations of the present invention are covered by the above teachings without departing from the spirit and intended scope of the invention.

1. An medical device comprising a surface region selected from (a) a superhydrophobic surface region and (b) a superhydrophilic surface region having a durometer of at least 40 A.

2. The medical device of claim 1, comprising a superhydrophobic surface region.

3. The medical device of claim 2, comprising two or more superhydrophobic surface regions.

4. The medical device of claim 2, wherein said superhydrophobic surface region has advancing and receding water contact angles of 150° or greater.

5. The medical device of claim 2, wherein said superhydrophobic surface region has advancing and receding water contact angles of 160° or greater.

6. The medical device of claim 2, wherein said superhydrophobic surface region corresponds to a tissue contacting surface of said medical device.

7. The medical device of claim 2, wherein said superhydrophobic surface region corresponds to the surface of a fluid conduit within said medical device.

8. The medical device of claim 7, wherein said fluid conduit is a cylindrical lumen with a diameter less than 250 micrometers.

9. The medical device of claim 7, wherein said fluid conduit is an annular space with an annular spacing less than 250 micrometer.

10. The medical device of claim 7, wherein said fluid conduit is a conduit for bodily fluid.

11. The medical device of claim 7, wherein said fluid conduit is a conduit for non-bodily fluids.

12. The medical device of claim 2, wherein said device is an internal medical device.

13. The medical device of claim 2, wherein said device is a balloon catheter.

14. The medical device of claim 13, wherein said superhydrophobic surface region lines an inflation lumen for said balloon.

15. The medical device of claim 14, wherein inflation fluid flowing by said superhydrophobic surface region has a slip length of at least 10 μm.

16. The medical device of claim 14, wherein inflation fluid flowing by said superhydrophobic surface region has a slip length of at least 50 μm.

17. The medical device of claim 13, wherein said superhydrophobic surface region is configured to contact a medical article.

18. The medical device of claim 17, wherein said medical article is a guidewire and said superhydrophobic surface region lines a guidewire lumen within said balloon catheter.

19. The medical device of claim 2, wherein said medical article is a stent and said superhydrophobic surface region is a balloon surface.

20. The medical device of claim 2, wherein said device is selected from a vascular catheter, a urinary catheter, a hydrolyser catheter, a guide wire, a pullback sheath, a left ventricular assist device, an endoscope, an airway tube and an injection needle.

21. The medical device of claim 2, wherein said superhydrophobic surface region is a textured fluorocarbon material surface.

22. The medical device of claim 2, wherein said superhydrophobic surface region is a textured fluorocarbon polymer surface.

23. The medical device of claim 22, wherein said surface region is textured using a laser ablation technique or a plasma etching technique.

24. The medical device of claim 2, wherein said superhydrophobic surface region corresponds to a coating formed over an underlying substrate.

25. The medical device of claim 24, wherein said coating is a multilayer coating.

26. The medical device of claim 24, wherein said coating comprises a fluorocarbon polymer layer.

27. The medical device of claim 26, wherein said fluorocarbon polymer layer is provided over a textured surface.

28. The medical device of claim 26, wherein said fluorocarbon polymer coating is deposited by a process selected from chemical vapor deposition and glow discharge deposition.

29. The medical device of claim 25, wherein said multilayer coating comprises a particulate layer.

30. The medical device of claim 29, wherein said particulate layer is a carbon nanotube layer.

31. The medical device of claim 25, wherein said multilayer coating comprises multiple layers of alternating charge.

32. The medical device of claim 31, wherein said layers of alternating charge comprise a negatively charged polyelectrolyte-containing layer, a positively charged polyelectrolyte-containing layer, and a charged particle layer.

33. The medical device of claim 31, wherein said multilayer coating comprises a fluorinated polyelectrolyte.

34. The medical device of claim 24, wherein said coating comprises a sol-gel layer.

35. The medical device of claim 34, wherein said coating further comprises an inherently hydrophobic layer over said sol-gel layer.

36. The medical device of claim 1, comprising a superhydrophilic surface region having a durometer of at least 40 A.

37. The medical device of claim 36, comprising two or more superhydrophilic surface regions having a durometer of at least 40 A.

38. The medical device of claim 36, wherein said superhydrophilic surface region has a static water contact angle of 2° or less.

39. The medical device of claim 36, wherein said superhydrophilic surface region has a static water contact angle of 1° or less.

40. The medical device of claim 36, wherein said superhydrophilic surface region corresponds to a tissue contacting surface of said medical device.

41. The medical device of claim 36, wherein said superhydrophilic surface region is configured to contact a medical article.

42. The medical device of claim 36, wherein said device is a balloon catheter.

43. The medical device of claim 36, wherein said device is selected from a vascular catheter, a urinary catheter, a hydrolyser catheter, a guide wire, a pullback sheath, an endoscope, an airway tube and an injection needle.

44. The medical device of claim 36, wherein said superhydrophilic surface region corresponds to a superhydrophilic coating over an underlying substrate.

45. The medical device of claim 44, wherein said coating is a multilayer coating.

46. The medical device of claim 45, wherein said coating comprises layers of alternating charge.

47. The medical device of claim 46, wherein said layers of alternating charge comprise a negatively charged polyelectrolyte-containing layer and a positively-charged polyelectrolyte containing layer.

48. The medical device of claim 47, wherein said layers of alternating charge further comprise charged particles.

49. The medical device of claim 36, wherein said device is an internal medical device.
